(19) United States
(12) Patent Application Publication
(10) Pub. No.: US 2015/0105643 A1
(43) Pub. Date: Apr. 16, 2015

(54) Intelligent Temperature Sensing Apparatus

(71) Applicant: Chen-Yu Chung, Taichung City (TW)

(72) Inventor: Chen-Yu Chung, Taichung City (TW)

(21) Appl. No.: 14/054,915

(22) Filed: Oct. 16, 2013

(51) Int. Cl. A61B 5/01; A61B 5/00; A61B 5/145; A61B 5/024; A61B 5/021

(52) U.S. Cl. CPC A61B 5/01; A61B 5/024; A61B 5/021; A61B 5/14532; A61B 5/6817; A61B 5/0022; H04W 88/02
USPC 600/365; 600/549; 600/508; 600/485

(57) ABSTRACT

An intelligent temperature sensing apparatus contains a temperature sensor having a first end for inserting into at least one ear and a second end for connecting with a smartphone; the smartphone in which a control software is loaded and saved; the control software for controlling a processor, an editor, and a storage device. The processor connects with the temperature sensor so as to process at least one measured temperature transmitted from the control software, the editor is used to edit/transform the at least one measured temperature, and the store device is served to record and store the at least one measured temperature.

### FIELD OF THE INVENTION

The present invent relates to an intelligent temperature sensing apparatus which is simplified and is portable easily.

### BACKGROUND OF THE INVENTION

A convention ear thermometer is applied to measure temperature, but it cannot match with a mobile device. An improved ear thermometer has been developed to cooperate with the mobile device; however, it has to be coupled with a circuit board of the mobile device, thus having complicated structure.

The present invention has arisen to mitigate and/or obviate the afore-described disadvantages.

### SUMMARY OF THE INVENTION

The primary object of the present invention is to provide an intelligent temperature sensing apparatus which is simplified and is portable easily.

To obtain the above objective, intelligent temperature sensing apparatus contains:

a temperature sensor having a first end for inserting into at least one ear and a second end for connecting with a smartphone;

the smartphone in which a control software is loaded and saved; and

the control software for controlling a processor, an editor, and a storage device.

The processor connects with the temperature sensor so as to process at least one measured temperature transmitted from the control software, the editor is used to edit/transform the at least one measured temperature, and the store device is served to record and store the at least one measured temperature.

Preferably, the temperature sensor transmits message in a wireless manner, for example, the temperature sensor transmits the message by means of a Bluetooth device.

It is preferable that the smartphone is replaced by any one of a laptop computer and a tablet computer.

In addition, the temperature sensor is replaced by any one of a pulse measuring device, a blood pressure measuring device, and a blood sugar measuring device.

### BRIEF DESCRIPTION OF THE DRAWINGS

FIG. 1 is a perspective view showing the assembly of an intelligent temperature sensing apparatus according to a preferred embodiment of the present invention.

FIG. 2 is a plan view showing the assembly of the intelligent temperature sensing apparatus according to the preferred embodiment of the present invention.

FIG. 3 is a flow chart showing the operation of the intelligent temperature sensing apparatus according to the preferred embodiment of the present invention.

### DETAILED DESCRIPTION OF THE PREFERRED EMBODIMENTS

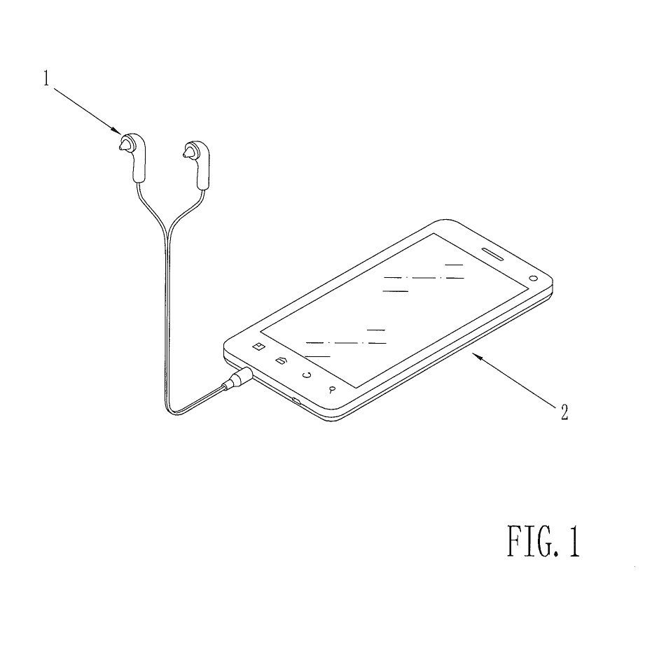

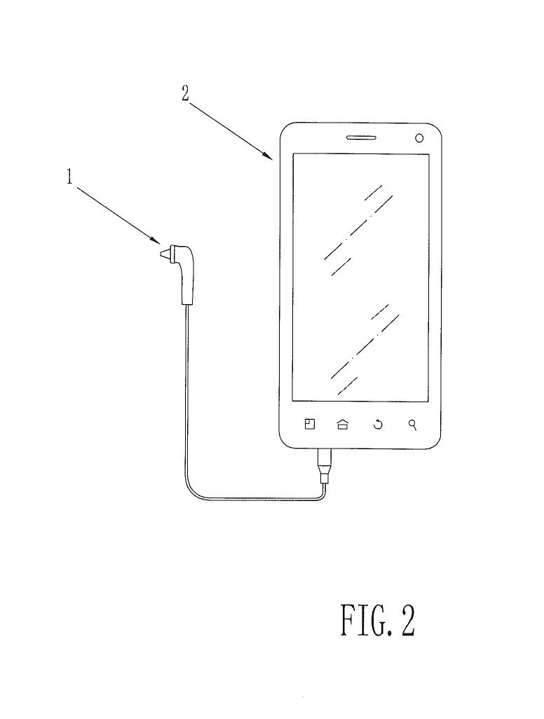

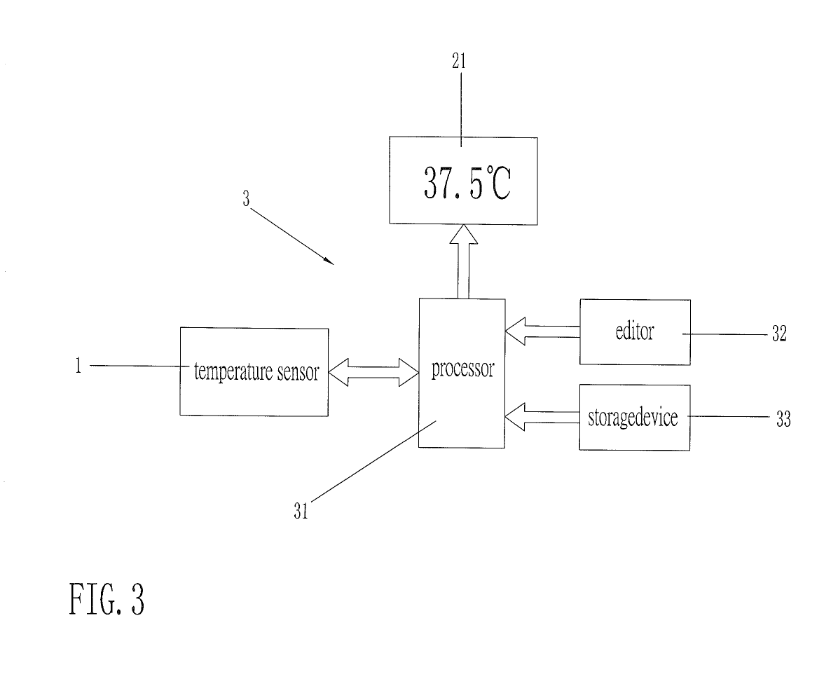

With reference to FIGS. 1-3, an intelligent temperature sensing apparatus according to a preferred embodiment of the present invention comprises:

a temperature sensor 1 having a first end for inserting into at least one ear and a second end for connecting with a smartphone 2;

the smartphone 2 in which a control software 3 is loaded and saved;
the control software 3 for controlling a processor 31, an editor 32, and a storage device 33; wherein the processor 31 connects with the temperature sensor 1 so as to process at least one measured temperature transmitted from the control software 3, the editor 32 is used to edit/transform the at least one measured temperature, and the store device 33 is served to record and store the at least one measured temperature.

Preferably, the temperature sensor 1 transmits message in a wireless manner, for example, the temperature sensor 1 transmits the message by means of a Bluetooth device. It is preferable that the smartphone is replaced by any one of a laptop computer and a tablet computer. In addition, the temperature sensor 1 is replaced by any one of a pulse measuring device, a blood pressure measuring device, and a blood sugar measuring device.

In operation, the control software 3 saved in the smartphone 2 is started to measure a temperature of the at least one ear and to control a measuring time (such as one second or a few seconds), and then the control software 3 transmits the at least one measured temperature to the processor 31 so that the processor 31 calculates an average value of the at least one measured temperature, thereafter the editor 32 edits/transforms the average value of the at least one measured temperature to/into a display message which will be displayed on a display screen 21, wherein the storage deceive 33 records and stores the average value of the at least one measured temperature, thus measuring temperature easily. Furthermore, the intelligent temperature sensing apparatus is portable conveniently. It is preferable that the control software is loaded and saved in the smartphone so as to measure temperature immediately.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. An intelligent temperature sensing apparatus comprising:
a temperature sensor having a first end for inserting into at least one ear and a second end for connecting with a smartphone;
the smartphone in which a control software is loaded and saved;
the control software for controlling a processor, an editor, and a storage device;
wherein the processor connects with the temperature sensor so as to process at least one measured temperature transmitted from the control software, the editor is used to edit/transform the at least one measured temperature, and the store device is served to record and store the at least one measured temperature.

2. The intelligent temperature sensing apparatus as claimed in claim 1, wherein the temperature sensor transmits message by means of a Bluetooth device.

3. The intelligent temperature sensing apparatus as claimed in claim 1, wherein the smartphone is replaced by any one of a laptop computer and a tablet computer.

4. The intelligent temperature sensing apparatus as claimed in claim 1, wherein the temperature sensor is replaced by any one of a pulse measuring device, a blood pressure measuring device, and a blood sugar measuring device.
